SEQUENCE LISTING (8 sequences)

SEQ ID NO: 1 - protein, 324 aa, Streptomyces roseochromogenus subsp. oscitans
MPALPIDQEFDCERFRADIRATAAAIGAPIAHRLTDTVLEAFRDNFAQGATLWKTTSQPGDQLSYRFFSRLKMDTVSRAIDAGLLDAAHPTLAVVDAWSSLYGGAPVQSGDFDAGRGMAKTWLYFGGLRPAEDILTVPALPASVQARLKDFLALGLAHVRFAAVDWRHHSANVYFRGKGPLDTVQFARIHALSGSTPPAAHVVEEVLAYMPEDYCVAITLDLHSGDIERVCFYALKVPKNALPRIPTRIARFLEVAPSHDVEECNVIGWSFGRSGDYVKAERSYTGNMAEILAGWNCFFHGEEGRDHDLRALHQHTESTMGGAR

SEQ ID NO: 2 - protein, 424 aa, Aspergillus terreus
MLPPSDSKDPRPWQILSQALGFPNYDQELWWQNTAETLNRVLEQCDYSVHLQYKYLAFYHKYILPSLGPFRRPGVEPEYISGLSHGGHPLEISVKIDKSKTICRLGLQAIGPLAGTARDPLNSFGDRELLKNLATLLPHVDLRLFDHFNAQVGLDRAQCAVATTKLIKESHNIVCTSLDLKDGEVIPKVYFSTIPKGLVTETPLFDLTFAAIEQMEVYHKDAPLRTALSSLKDFLRPRVPTDASITPPLTGLIGVDCIDPMLSRLKVYLATFRMDLSLIRDYWTLGGLLTDAGTMKGLEMVETLAKTLKLGDEACETLDAERLPFGINYAMKPGTAELAPPQIYFPLLGINDGFIADALVEFFQYMGWEDQANRYKDELKAKFPNVDISQTKNVHRWLGVAYSETKGPSMNIYYDVVAGNVARV

SEQ ID NO: 3 - protein, 311 aa, Streptomyces rubidus
MPGASKTEAVYAAIEEASGLLGVPCSREKVQQVLGAFGEGVSEESVVVLAMAGGERYRGDIDYNFTVPTEIGDPYKIAVANGLLEADDHPAGRLGADIAEHCRVSFFGVEAGVVGGFKKTYVFFPLDELGSLSKLAEIDSMPPSVAEHAAAFARNGMDDRISIVGIDYLSQTMNVYFMAAPVDQKLALDLLEDLELPAPSPDLLEFIPNSFSIYPTYSWDSSQIKRICFSAVSPDQSAYPTTLHPEIATFARNAPYEYGGARVLVYGATITRGEEYHKLGAYFRRPAAFWNSLPLAATFERLAAEQAAAGK

SEQ ID NO: 4 - protein, 313 aa, Aspergillus calidoustus
MTVIEPPAPVPIATPFSQSRFLSELESACKALNTAYSEPTTLKILKTFEKSFTRGAVLFRTTNQPEGPLNYRVYERQSIDMVSLATNAGLLPRDSSLGKLVLTWSSLYGNESHQLADFDSSKGLVKFWVFLGGIRPLDEILNVPGVPECIRGYRGMFKTINLTGVRHTAVDLEKGSMNLYFRTPPDLSRRDMDEYISLAGADPLPDALFEEIHAPFPRAGGTFAVTMDMTTGRISRVAFYALRLEENNPPASAVSGRIKTFFEVCPSYDPDEMKALAWSFGEGNKRYVKAEHSYTGDLVGLIRGWGTSMSCAE

SEQ ID NO: 5 - protein, 305 aa, Unknown
  uncultured bacterium esnapd16.1
MPERSALEEIYSAIDTTARVLDVPCDRDKVQPVLNAYGDGIASGLMVFTMAAGESRRGDFDYNFTVPADGPDPYAVAVANGFTPRTDHPVGSLLSDIAERSPLQFYGAECGVVEGFKKTYAFFPLDDLQRASKLADIPSVPPSLGENLSLLAGYGLDDNVSIVGIDYVHRTMNVYFGRFSADHIEPGTVRSLLRDIGMPELSDEMLGFTRNAFSIYPTFSWDSPGITRICFSLVTQDPRALPAVLEPEIGRFVRNTPYAYEGEDDRILVYGATLSARGEYWKAGVYHRRPAEYWSKVQLFDKIVN

SEQ ID NO: 6 - protein, 300 aa, Streptomyces cinnamonensis
MMSGTADLAGVYAAVEESAGLLDVSCAREKVWPILAAFEDVLPTAVIAFRVATNARHEGEFDCRFTVPGSIDPYAVALDKGLTHRSGHPIETLVADVQKHCAVDSYGVDFGVVGGFKKIWVYFPGGRHESLAHLGEIPSMPPGLAATEGFFARYGLADKVDLIGVDYASKTMNVYFAASPEVVSAPTVLAMHREIGLPDPSEQMLDFCSRAFGVYTTLNWDSSKVERIAYSVKTEDPLELSARLGSKVEQFLKSVPYGIDTPKMVYAAVTAGGEEYYKLQSYYQWRTDSRLNLSYIGGRS

SEQ ID NO: 7 - protein, 380 aa, Clostridium clariflavum
MSEQVCEILWSKKMQEYFNSLTPNMPEGLRDRYIGYVNENARARGASEVTELDIITALLRNTPVFFRQNISNIPKDIKVDLSSLTKRDLALMSDDIEKAAKIAEVGCNKELVNKILNVYEEQFSHPQVAVSFRTTTKPVEKRSLDVRYVDVWTSHDPYAMAIENGLLVKSGHPVDNLFYDIKSNFPIMGYGVDFGVLNGFAKIWMRIPTHLPVPLEKLQLIPSFPDSLKNYISLLSKYSMDRIIMVGIDYIHKSTNIYFVKRYYGDLSTETVTSLISELGFPVPSNELIEECAHALFFYCTFTWDSPKIERLSFHRVAHDQSQVPVHLHPFLEKYSLNAPILGDKRKFMYTVALSPKGNYIKLESDYSSGIMADALVETY

SEQ ID NO: 8 - protein, 292 aa, Nocardia brasiliensis
MTLDNLRRDLREYARLAEVGYDPAVVDPVLEPLADLWTNSVVAVRTTTHPVPERDVNVRLMHAGEPTGLVTTLRDAGLITYTGHPMEELLAAVSAAVPARAGVDVALSEGVQKIWLIFPELLSVERMLAFPGVPEAARAHAAHLSRYGGRIGILAVDFAARTMNLYSNVFEPGSLGSADIAEILADLDFTAATEEELALLGRTFNIYRTFSWTSARMQRICFPLRVQAANFPAHLHPVLARFVEGAPFVDPDTRGFVFYAAYGPTDRYYKVQAEYATGQPVTFPGGTAPRVN

(19) United States
(12) Patent Application Publication
(10) Pub. No.: US 2024/0084337 A1
(43) Pub. Date: Mar. 14, 2024

(54) BIOSYNTHESIS OF CANNABINOID PRECURSORS USING NOVEL AROMATIC PRENYL TRANSFERASES

(71) Applicant: National University of Singapore, Singapore (SG)

(72) Inventors: Maybelle Darlene Kho Go, Singapore (SG); Kevin Jie Han Lim, Singapore (SG); Yan Ping Lim, Singapore (SG); Wen Shan Yew, Singapore (SG)

(21) Appl. No.: 17/767,722

(22) PCT Filed: Oct. 12, 2020
(86) PCT No.: PCT/SG2020/050582

§ 371 Date: Apr. 8, 2022

Related U.S. Application Data
(60) Provisional application No. 62/913,933, filed on Oct. 11, 2019.

(51) Int. Cl. C12P 7/42; C12N 9/10; C12N 15/81

(52) U.S. Cl. CPC C12P 7/42; C12N 9/1085; C12N 15/81; C12Y 205/01039

(57) ABSTRACT

A method for producing a cannabinoid precursor by contacting a substrate and geranyl pyrophosphate or farnesyl pyrophosphate with an NphB orthologue. The NphB orthologue is from an organism other than Cannabis sativa, and the substrate can be 2,4-dihydroxy-6-pentylbenzoic acid or 2,4-dihydroxy-6-propylbenzoic acid. Also disclosed is a recombinant cell of Yarrowia lipolytica, carrying in its genome a nucleic acid encoding an NphB orthologue from an organism other than Cannabis sativa such that the NphB orthologue is expressed in the recombinant cell.

### BACKGROUND

The structure and function of an aromatic prenyl transferase (NphB) from Streptomyces sp. strain CL190 has been elucidated. See Kumano et al., Bioorg. Med. Chem. 2008, 16(17):8117-26. Previous studies reported in Zirpel et al., J. Biotechnol. 2017, 259:204-212, showed that NphB was able to utilize the same substrates as the membrane-bound geranylpyrophosphate: olivetolate geranyltransferase from the Cannabis plant to form the cannabinoid precursor cannabigerolic acid (CBGA).

CBGA is commonly known as the first branch point of the cannabinoid biosynthetic pathway found in the Cannabis plant, and wild-type NphB can make CBGA, as well as an O-prenylated side product, i.e., 2-O-geranyl olivetolic acid.

Further enzymes and methods are needed for synthesizing known cannabinoid precursors and novel cannabinoid precursors with specificity and high yield.

### SUMMARY

A method is disclosed for producing a cannabinoid precursor. The method includes the steps of contacting a substrate and geranyl pyrophosphate or farnesyl pyrophosphate with an NphB orthologue. The substrate can be, e.g., 2,4-dihydroxy-6-pentylbenzoic acid or 2,4-dihydroxy-6-propylbenzoic acid, and the NphB orthologue is from an organism other than Cannabis sativa.

Also provided is a recombinant cell of Yarrowia lipolytica, carrying in its genome a nucleic acid encoding an NphB orthologue. The NphB orthologue, which is from an organism other than Cannabis sativa, is expressed in the recombinant cell.

The details of one or more embodiments are set forth in the description and the examples below. Other features, objects, and advantages will be apparent from the detailed description, from the drawings, and also from the appended claims.

### BRIEF DESCRIPTION OF THE DRAWINGS

The invention description below refers to the accompanying drawings, of which:

FIG. 1 shows the structures of cannabinoid products using olivetolic acid and geranyl pyrophosphate as substrate. The molecular formula and formula weight of all the possible products are C₂₂H₃₁O₄ and 359.22, respectively. Black: part of olivetolic acid which is derived from three units of malonyl-CoA; Dark Gray: part of olivetolic acid derived from hexanoyl-CoA; Light Gray: part of geranyl pyrophosphate which is transferred by the orthologues.

FIG. 2 is a diagram of the pYLEX1 vector used to integrate genes encoding NphB orthologues into the Y. lipolytica genome.

FIG. 3A shows LC-MS spectra of cannabinoid precursors having m/z=359.22 biosynthesized from three NphB orthologues: row 1=CBGA standard, row 2=P3E2, row 3=no enzyme negative control, row 4=P3A5, row 5=P3E8, row 6=NphB positive control, row 7=1BF1, row 8=Y1C5. Peaks are identified by retention times and relative area under the peak.

FIG. 3B shows LC-MS spectra of cannabinoid precursors having m/z=359.22 biosynthesized from three additional NphB orthologues: row 1=10 μg/ml CBGA standard, row 2=no enzyme negative control, row 3=NphB positive control, row 4=P3F5, row 5=P3A6, row 6=1BC2. Peaks are identified by retention times and relative area under the peak.

FIG. 4 shows the structures of potential cannabinoid products biosynthesized using divarinic acid (top row), which has two fewer carbon units compared to olivetolic acid, resulting in the production of CBGVA, which is two carbons shorter than CBGA, or using farnesyl pyrophosphate (bottom row) instead of geranyl pyrophosphate resulting in the production of a novel cannabinoid precursor.

### DETAILED DESCRIPTION

Disclosed are enzymes that catalyze the biosynthesis of cannabinoid precursors by transferring isoprene units from certain pyrophosphates, e.g., geranyl pyrophosphate, to aromatic polyketides such as 2,4-dihydroxy-6-pentylbenzoic acid, i.e., olivetolic acid, and 2,4-dihydroxy-6-propylbenzoic acid. These enzymes, from organisms other than Cannabis sativa, can be recombinantly expressed in Escherichia coli or Yarrowia lipolytica and subsequently employed for their prenyl transferase activity.

As summarized above, a method for producing a cannabinoid precursor is disclosed. In a specific example, the substrate is olivetolic acid, the pyrophosphate is geranyl pyrophosphate, and the cannabinoid precursor produced has a mass to charge ratio of 359.22 and a retention time of longer than 6.1 minutes, as determined by LC/MS analysis. The cannabinoid precursor described herein also falls within the scope of the invention.

In the above method, the source of the NphB orthologue can be, but is not limited to, Streptomyces roseochromogenus subsp. oscitans, Streptomyces rubidus, Streptomyces cinnamonensis, Aspergillus calidoustus, Aspergillus terreus, Clostridium clariflavum, Nocardia brasiliensis, and uncultured bacterium esnapdl6.1.

In a particular aspect of the method, the NphB orthologue has the amino acid sequence of any one of SEQ ID NOs: 1-8. Alternatively, the NphB orthologue can have an amino acid sequence at least 70% identical (e.g., at least 70%, 75%, 80%, 85%, 90%, 95%, and 99%) to any one of SEQ ID NOs: 1-8 and has aromatic prenyl transferase activity.

In an exemplary method, the NphB orthologue is a recombinant enzyme. The recombinant enzyme can be produced in, e.g., Escherichia coli and Yarrowia lipolytica.

Also mentioned above is a recombinant cell of Yarrowia lipolytica, including in its genome a nucleic acid encoding an NphB orthologue. The NphB orthologue is from an organism other than Cannabis sativa.

Exemplary sources of the NphB orthologue are Streptomyces roseochromogenus subsp. oscitans, Streptomyces rubidus, Streptomyces cinnamonensis, Aspergillus calidoustus, Aspergillus terreus, Clostridium clariflavum, Nocardia brasiliensis, and uncultured bacterium esnapd16.1.

In a particular recombinant cell, the NphB orthologue has the amino acid sequence of any one of SEQ ID NOs: 1-8. In another example, the NphB orthologue can have an amino acid sequence at least 70% identical (e.g., at least 70%, 75%, 80%, 85%, 90%, 95%, and 99%) to any one of SEQ ID NOs: 1-8 and has aromatic prenyl transferase activity.

Without further elaboration, it is believed that one skilled in the art can, based on the disclosure herein, utilize the present disclosure to its fullest extent. The following specific example is, therefore, to be construed as merely descriptive, and not limitative of the remainder of the disclosure in any way whatsoever. All publications cited herein are incorporated by reference in their entirety.

### EXAMPLE

Through a search of available sequence databases, 105 gene orthologues to NphB were identified that were also annotated in the UniProt database as potential aromatic prenyl transferases. The genes were synthesized and cloned into a modified pES2 vector containing a T7 promoter and terminator. The vectors were transformed into Escherichia coli Acella™ cells using chemical treatment and grown on LB+streptomycin selection plates at 37° C. Sequence-verified clones were picked and grown in LB+streptomycin media until an OD₆₀₀ₙₘ of 0.8. Protein expression was induced by adding 0.1 mM IPTG to the cultures and incubating at 25° C. for 24 h. The cells were subsequently harvested and stored at −20° C. until protein purification.

Pelleted cells were resuspended in 100 μL of binding buffer containing 20 mM Tris-HCl (pH 7.9), 500 mM NaCl, and 5 mM imidazole. The cells were subsequently lysed by sonication and the cell debris was removed by centrifugation for 30 min. at 4° C. The supernatant containing His-tagged proteins was purified using Ni²⁺ affinity chromatography and the recombinant proteins were eluted with buffer containing 20 mM Tris-HCl (pH 7.9), 500 mM NaCl, and 100 mM L-histidine. The concentration of His-tagged proteins was estimated using an ELISA detection kit, and the purified NphB orthologues were stored at 4° C.

The NphB orthologues were tested for prenyl transferase using an in vitro prenyl transferase assay. In a reaction volume of 200 μL, 20 μL of 1 M Tris-HCl (pH 7.9), 2 μL of 1 M MgCl₂, 4 μL of 50 mM aromatic polyketide substrate, e.g., olivetolic acid and divarinic acid, 20 μL of 10 mM geranyl pyrophosphate, and 50 μg of purified NphB orthologues were combined and incubated at 30° C. A control reaction without enzyme was also prepared.

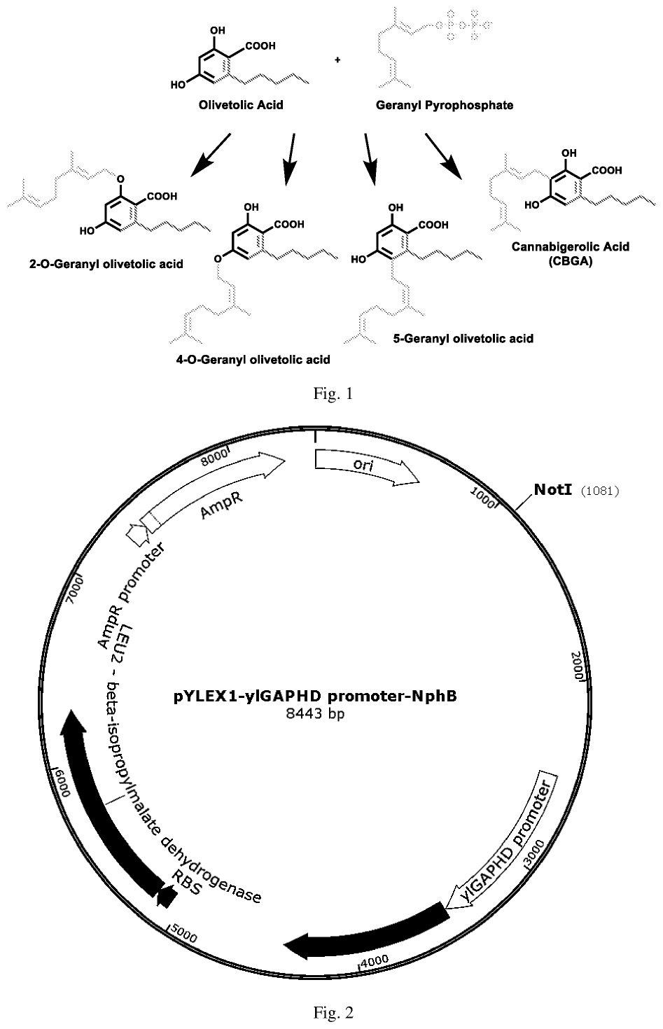

After 24 h, the reaction mixture was acidified to pH 3.0 with 6 M HCl and extracted with ethyl acetate three times. The samples were dried under vacuum and redissolved in methanol for LC-MS analysis using the negative ion mode. Extracted-ion-chromatograms (EIC) for m/z=359.22 (if olivetolic acid is used) and m/z=331.19 (if divarinic acid is used) were generated for each sample to determine if the biosynthesis of a potential cannabinoid precursor was catalyzed by the NphB orthologue. The structures of potential cannabinoid precursors that can be biosynthesized using olivetolic acid and geranyl pyrophosphate as substrates are shown in FIG. 1.

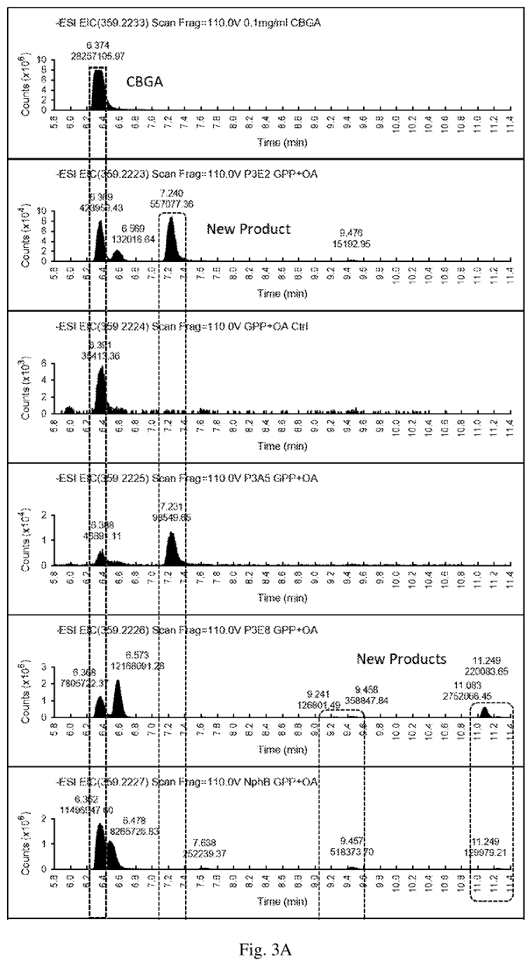

Certain orthologues of NphB produced a novel product, identified as a new peak in LC-MS analysis compared to the peaks formed by wild-type NphB. Particular orthologues also demonstrated a greater yield of CBGA. Such orthologues are selected for subsequent cloning into Yarrowia lipolytica. They are subcloned into a modified pYLEX1 vector (Yeastern Biotech; see FIG. 2) and transformed into Y. lipolytica using chemical treatment. Transformants are selected on YNB agar without leucine and used for subsequent yield optimization assays.

Out of 105 orthologues tested, eight orthologues showed good protein expression and novel prenyl transferase activities. The eight orthologues are listed in Table 1 below, together with corresponding Uniprot ID and source organism.

TABLE 1
NphB orthologues having novel prenyl transferase activity.

| | | | % | |
|---|---|---|---|---|
| | | | identity | SEQ |
| Uniprot ID | Orthologue | Organism | to NphB | ID NO: |
| Q8GHB2 | P3A5 | Streptomyces | 14.3 | 1 |
| | | roseochromogenus | | |
| | | subsp. oscitans | | |
| A0A1B0UHJ4 | Y1C5 | Aspergillus terreus | 11.7 | 2 |
| A0A1H8R5X4 | P3E8 | Streptomyces rubidus | 36.2 | 3 |
| A0A0U5C5V3 | P3E2 | Aspergillus calidoustus | 14.0 | 4 |
| S5UCZ5 | 1BF1 | uncultured bacterium | 33.8 | 5 |
| | | esnapd16.1 | | |
| A2AXG5 | P3A6 | Streptomyces | 35.7 | 6 |
| | | cinnamonensis | | |
| G8LVY5 | P3F5 | Clostridium clariflavum | 25.4 | 7 |
| K0EWY8 | 1BC2 | Nocardia brasiliensis | 16.1 | 8 |

P3E8 had a comparable yield of CBGA compared to wild-type NphB, and all eight orthologues showed at least one new peak in LC-MS analysis which also had a molecular formula of C₂₂H₃₁O₄(FW=359.22).

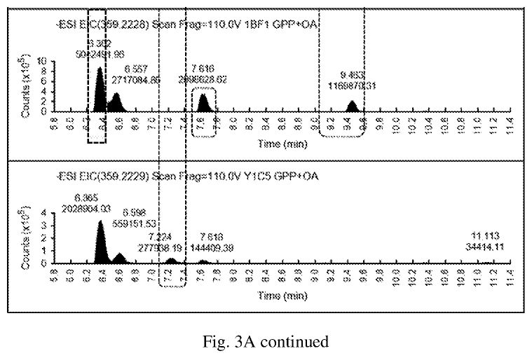

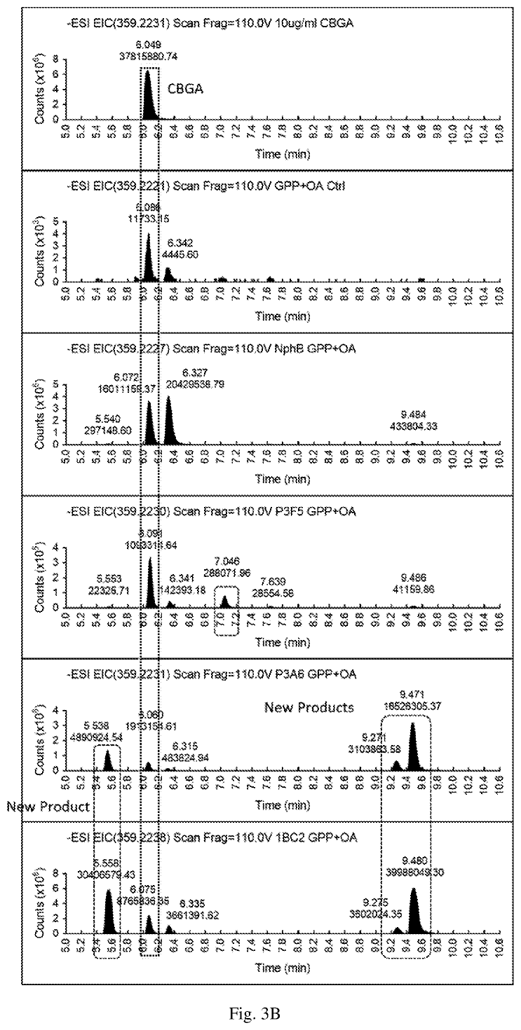

As shown in FIGS. 3A and 3B, all eight orthologues, as well as wild-type NphB, can produce CBGA (retention time: 6.4 min. in FIG. 3A and 6.1 min. in FIG. 3B). All orthologues except for P3A5 also produced a side product, i.e., 2-O-geranyl olivetolic acid (retention time: 6.6 min. in FIG. 3A and 6.3 min. in FIG. 3B). Orthologues P3A5, Y1C5, and P3E2 each produced significant amounts of a new prenylated product with m/z=359.2225 at a retention time of 7.23-7.24 min. that was not observed in the reaction using wild-type NphB.

Novel products having m/z=359.22 and identified in FIGS. 3A and 3B are summarized in Table 2 below. These products have retention times not seen for those produced with the wild-type NphB enzyme.

TABLE 2
Novel products produced by orthologues from
geranyl pyrophosphate and olivetolic acid

| | Retention time(s) |
|---|---|
| Orthologue | in min. |
| P3E2 | 7.24 |
| P3A5 | 7.23 |
| P3E8 | 9.24, 11.08 |
| 1BF1 | 7.62, 9.46 |
| Y1C5 | 7.22, 7.62 |
| P3F5 | 7.05 |
| P3A6 | 5.54, 9.27, 9.47 |
| 1BC2 | 5.56, 9.28, 9.48 |

A study showed that two orthologues, corresponding to Uniprot IDs: C4PWA1 and Q9L9F1, produced only trace amounts of new prenylated products, suggesting that these orthologues had prenylated the olivetolic acid at a site different from wild-type NphB.

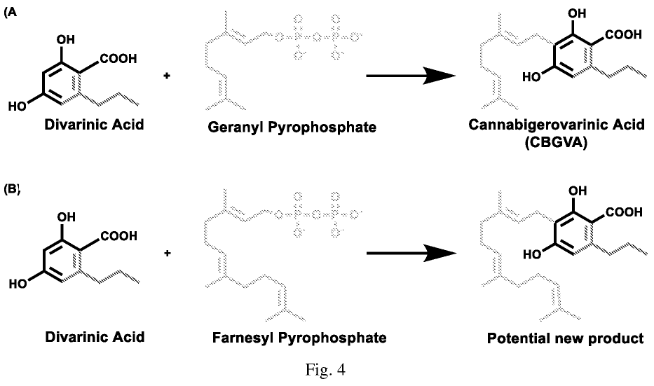

In addition, different substrates can be incubated with the NphB orthologues to determine if novel cannabinoid precursors can be produced. FIG. 4 shows the potential product that can be formed when either olivetolic acid or geranyl pyrophosphate, or both, are replaced. Novel cannabinoid precursors can then be tested with downstream cannabinoid synthases in order to diversify cannabinoid compound libraries.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method for producing a cannabinoid precursor, the method comprising contacting a substrate and a pyrophosphate selected from geranyl pyrophosphate and farnesyl pyrophosphate with an NphB orthologue, wherein the substrate is 2,4-dihydroxy-6-pentylbenzoic acid (olivetolic acid) or 2,4-dihydroxy-6-propylbenzoic acid and the NphB orthologue is from an organism other than Cannabis sativa.

2. The method of claim 1, wherein the substrate is olivetolic acid, the pyrophosphate is geranyl pyrophosphate, and the cannabinoid precursor has a mass to charge ratio of 359.22 and a retention time of longer than 6.1 minutes, as determined by LC/MS analysis.

3. The method of claim 2, wherein the NphB orthologue is from Streptomyces roseochromogenus subsp. oscitans, Streptomyces rubidus, Streptomyces cinnamonensis, Aspergillus calidoustus, Aspergillus terreus, Clostridium clariflavum, Nocardia brasiliensis, or uncultured bacterium esnapd16.1.

4. The method of claim 3, wherein the NphB orthologue is a recombinant enzyme produced in Yarrowia lipolytica.

5. The method of claim 1, wherein the NphB orthologue is from Streptomyces roseochromogenus subsp. oscitans, Streptomyces rubidus, Streptomyces cinnamonensis, Aspergillus calidoustus, Aspergillus terreus, Clostridium clariflavum, Nocardia brasiliensis, or uncultured bacterium esnapd16.1.

6. The method of claim 5, wherein the NphB orthologue is a recombinant enzyme produced in Yarrowia lipolytica.

7. The method of claim 1, wherein the NphB orthologue has the amino acid sequence of any one of SEQ ID NOs: 1-8 or an amino acid sequence at least 70% identical to any one of SEQ ID NOs: 1-8 and having aromatic prenyl transferase activity.

8. The method of claim 7, wherein the NphB orthologue is a recombinant enzyme produced in Yarrowia lipolytica.

9. The method of claim 2, wherein the NphB orthologue has the amino acid sequence of any one of SEQ ID NOs: 1-8 or an amino acid sequence at least 70% identical to any one of SEQ ID NOs: 1-8 that has aromatic prenyl transferase activity.

10. The method of claim 9, wherein the NphB orthologue is a recombinant enzyme produced in Yarrowia lipolytica.

11. A recombinant cell of Yarrowia lipolytica, comprising in its genome a nucleic acid encoding an NphB orthologue, wherein the NphB orthologue is from an organism other than Cannabis sativa, and the NphB orthologue is expressed in the recombinant cell.

12. The recombinant cell of claim 11, wherein the NphB orthologue is from Streptomyces roseochromogenus subsp. oscitans, Streptomyces rubidus, Streptomyces cinnamonensis, Aspergillus calidoustus, Aspergillus terreus, Clostridium clariflavum, Nocardia brasiliensis, or uncultured bacterium esnapd16.1.

13. The recombinant cell of claim 11, wherein the NphB orthologue has the amino acid sequence of any one of SEQ ID NOs: 1-8 or an amino acid sequence at least 70% identical to any one of SEQ ID NOs: 1-8 and having aromatic prenyl transferase activity.
